(19) United States
(12) Patent Application Publication
(10) Pub. No.: US 2011/0112519 A1
(43) Pub. Date: May 12, 2011

(54) Apparatus for cutting a tissue part with focused laser radiation

(76) Inventors: Christof Donitzky, Eckental (DE); Christian Wüllner, Mohrendorf (DE)

(21) Appl. No.: 12/972,899

(22) Filed: Dec. 20, 2010

(30) Foreign Application Priority Data
Jun. 20, 2008 (EP) PCT/EP2008/005012

(51) Int. Cl. A61F 9/009; A61B 18/20

(52) U.S. Cl. 606/4; 606/13

(57) ABSTRACT

An apparatus for cutting a tissue part out of a tissue by means of focused laser radiation, comprising a suction ring which has a sealing surface that is capable of being applied onto a surface of the tissue, devices for generating an underpressure in a cavity that is delimited by the sealing surface, by the surface of the tissue and by the suction ring, and an applanation plate that is capable of being pressed against the surface of the tissue for the purpose of shaping, and a body that is opaque to the laser radiation, that interacts with the suction ring and with its inner edge defines an edge of the tissue part.

This application claims priority to internationally filed application PCT/EP2008/005012 filed on Jun. 20, 2008.

The invention relates to an apparatus for cutting a tissue part out of a tissue with focused laser radiation. In particular, the apparatus relates to the cutting of a so-called flap out of the cornea of an eye in the course of refractive surgery, in particular LASIK. The invention will be elucidated in more detail in the following with regard to refractive surgery, in particular LASIK. But, in addition to this, the invention can also be employed generally for the purpose of cutting a tissue part out of a tissue by means of focused laser radiation.

In the case of LASIK—that is to say, the now generally known correction of the optical imaging properties of the cornea by means of laser radiation—it is known firstly to cut a so-called flap out of the anterior region of the cornea, whereby a portion of the flap remains connected to the cornea like a hinge, so that the flap can be folded aside for the subsequent ablation of corneal tissue by means of laser radiation. After implementation of the ablation (resection of tissue), the flap is folded back, and a relatively rapid healing takes place, the corneal surface being largely undamaged.

In the state of the art there are principally two different techniques for the production of the flap.

On the one hand, for the cutting of the flap a mechanical so-called microkeratome is employed which cuts into the cornea from the side with a rapidly oscillating cutting edge. In this case a so-called suction ring is mounted onto the eye, which fixes the eye by means of vacuum. This is the state of the art in this regard.

On the other hand, laser radiation is also increasingly being employed for the cutting of the flap—at present, focused laser radiation with pulse lengths within the femtosecond range. In this case the radiation is focused below the anterior surface of the cornea, within the tissue, and the focal points are positioned in the desired surface in such a way that, as a result, a flap is cut out of the cornea. This is well-known in the state of the art as femto LASIK.

With femto LASIK there are, in particular, two embodiment variants:

According to a first embodiment, a separate system consisting of suction ring and lid lock is employed for the purpose of lateral fixation of the eye. A planar (flat) optical plate is impressed for the purpose of levelling the cornea. The stroma is cut two-dimensionally at depth in the manner described above. The marginal incision (that is to say, the positioning of the focal points in the marginal region of the flap) is effected in this case by positioning of the focal points out of the plane as far as the corneal surface. In this case, the focal points reach into the aforementioned planar optical plate which, as a result, is destroyed and consequently constitutes a disposable article.

According to another embodiment of femto LASIK, the planar stromal incision is likewise carried out using a suction ring with an applanation by suction of the eye. In this case, the depth of the incision is defined by a plastic film that is transparent to the laser wavelength and that is situated between the applanation plate and the cornea. In this case too, the plastic film is a disposable article. A special configuration of the edge of the incision is not provided in this case.

The invention has as its objective to make an apparatus available with which a tissue part, in particular a flap, can be cut out of a tissue, in particular the cornea, in simple manner with focused laser radiation. In this connection, in particular the duration of the operation is to be shortened, and the energy introduced for the incision into the eye is to be reduced.

For this purpose the invention makes an apparatus available for cutting a tissue part out of a tissue by means of focused laser radiation, exhibiting the following:
a suction ring which has a sealing surface that is capable of being applied onto a surface of the tissue,
devices for generating an underpressure in a cavity that is delimited by the surface of the tissue and by the suction ring, and
applanation plate that is capable of being pressed against the surface of the tissue for the purpose of shaping, the apparatus being distinguished by
a body that is opaque to the laser radiation and that interacts with the suction ring and defines with its inner edge an edge of the tissue part.

According to the invention, in the case of femto LASIK, for example, for each desired flap diameter and for each desired geometry of the flap a “customised” body, opaque to the laser radiation, in the above sense is selected and is employed with the suction ring. In this case the aforementioned body that is opaque to the laser radiation may have been firmly connected to the suction ring, so that a specific suction ring is procured for the desired shape of the flap in the given case, or the body that is opaque to the laser radiation is detachably connected to the suction ring as an interchangeable part.

The ‘applanation plate’ in the sense of the present invention does not necessarily have to be planar (flat) but may also exhibit a curvature, for example a spherical curvature, depending on the desired incision.

The aforementioned body that is opaque to laser radiation is preferably annular.

Another configuration provides that the body that is opaque to the laser radiation either partly absorbs or entirely absorbs the laser radiation.

Another configuration of the invention provides that the body that is opaque to laser radiation rests on a contact plate which with a sealing surface is capable of being brought into contact with the tissue.

Exemplary embodiments of the invention will be elucidated in more detail in the following on the basis of the drawing. Shown are:

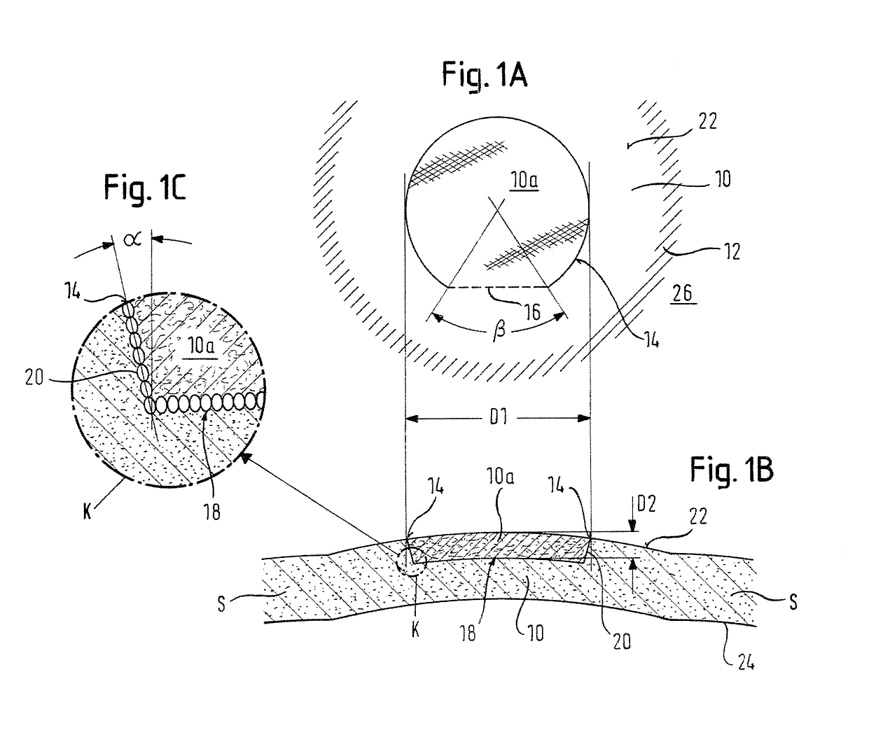

FIGS. 1A-1C: schematically, the production of a flap in the case of femto LASIK;

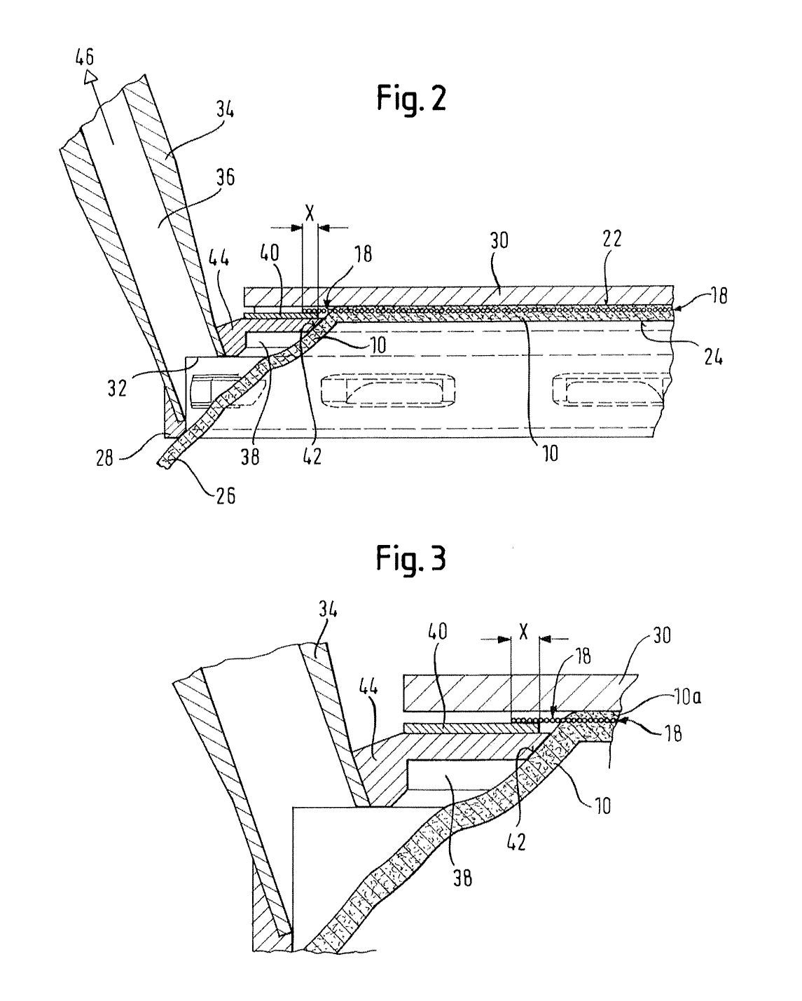

FIG. 2: schematically, an exemplary embodiment of an apparatus for cutting a tissue part out of tissue in accordance with the invention;

FIG. 3: a detail from FIG. 2 on an enlarged scale; and

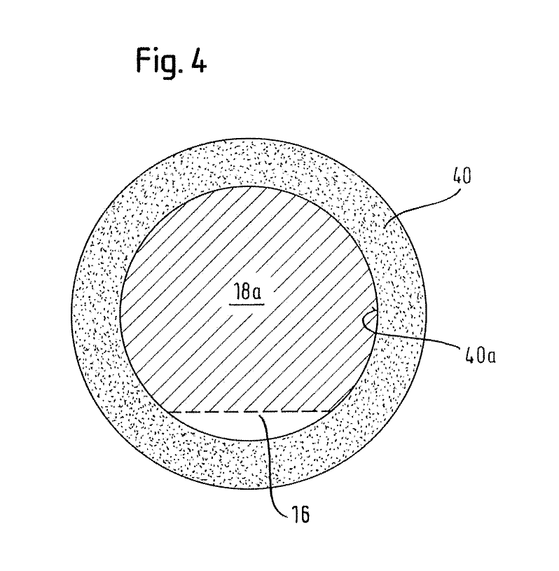

FIG. 4: a top view of a body that is opaque to laser radiation.

FIGS. 1A 1B and 1C show schematically the conditions in the course of the cutting of a flap 10a in a cornea 10 using focused radiation with pulse lengths within the femtosecond range. The laser wavelength that is used corresponds to the state of the art.

FIG. 1A show a top view of the cornea 10, the limbus 12 thereof, the edge 14 of the flap 10a and the sclera 26. In an articulated part 16 (hinge) the cut flap 10a remains connected to the cornea 10, so that it can be folded upwards in a manner known as such. FIG. 1B shows the focal plane 18 of the laser—that is to say, the plane in which the focal points of the laser radiation are positioned in such a way that an incision arises overall. In this connection the term ‘plane’ which is used here is not to be understood in technical language as meaning absolutely planar but also encompasses curved designs of the surface of the focal points, as the incision according to FIG. 1B shows, for example.

The circular detail K according to FIG. 1B is represented in FIG. 1C on an enlarged scale. According to this Figure, the surface in which the laser focal points are placed is pulled upwards in the marginal region in a lateral incision 20. At the points where the incision plane of the focal points intersects the anterior surface 22 of the cornea 10 the edge 14 of the cut flap is located. FIG. 1B also shows the diameter D1 and the thickness D2 of the flap and the posterior surface 24 of the cornea 10.

The lateral incision 20 and the corresponding guidance of the focal points of the laser radiation are elaborate and complicated, which also has consequences on the probability of imperfect incisions. The lateral incision at the edge 14 is also time-consuming and gives rise to a relatively high total energy that is introduced into the eye. Both are improved with an apparatus according to FIGS. 2 to 4.

FIG. 2 shows a suction ring 28 with an applanation plate 30 which is pressed onto the anterior surface 22 of the cornea 10. As already stated above, the applanation plate 30 does not have to be absolutely planar (flat) but may also be curved, for example spherical.

The suction ring 28 has, in a manner known as such, openings 32 for generating an effective but not excessive underpressure in the space 38 in such a manner that the eye is laterally fixed in the suction ring 28. The suction ring 28 further exhibits, in a manner known as such, a connection piece 34, in which a duct 36 leads to a vacuum pump (not shown) which is indicated by reference symbol 46.

The suction ring 28 exhibits a contact plate 44 which bears with a sealing surface 42 against the anterior surface 22 of the cornea 10, so that the space 38 is sealed against the cornea and in said space an underpressure prevails in relation to the external atmosphere.

The surface 18 in which the laser focal points are positioned is represented in FIGS. 2 and 3. This focal plane of the laser is totally planar in the exemplary embodiment that is represented. This holds, in particular, for the region at the edge 14 of the incision. For the purpose of defining the incision edge 14, use is made of a body 40 that is opaque to the laser radiation. FIG. 4 shows in top view an exemplary embodiment of the body 40 that is opaque to laser radiation. According to FIG. 2 the body 40 is placed onto the aforementioned suction-ring contact plate 44. It is situated below the applanation plate 30 and is chosen in such a way that its inner edge 40a (see FIG. 4) corresponds to the desired edge 24 of the flap 10a. Consequently, the shape of the flap 10a can be determined by selection of the shape of the body 40. According to FIG. 4 the laser pulses in region 18a become active—i.e. the laser incision is effected exclusively in the surface 18a which in FIG. 4 is drawn in cross-hatched manner, so that the hinge region 16 remains uncut. An angled lateral incision 20 with the angle α according to FIG. 1C is not necessary here. Rather, in the marginal region the guidance of the incision can remain planar. FIG. 2 shows the region ‘X’ in which the laser radiation can be guided, without disadvantages for the desired incision, over the body 40 that is opaque to laser radiation. This simplifies the control for the laser beam.

The exemplary embodiment according to FIG. 2 shows a plane 18 of the focal points of the laser which is planar in the strict sense, but inside the incision—that is to say, outside the region directly around the edge 14—the guidance of the incision may also be other than flat, for example spherical or such like.

The applanation plate 30 remains undamaged in the course of the operation. The thickness (see FIG. 1B, reference symbol D2) of the flap can be adapted to the desired shape of the incision by variation of the depth of the focal points in the stromal tissue. A change in the position of the focal points of the laser in the marginal region in the z-direction (ordinarily, by ‘z-direction’ here the direction perpendicular to the applanation plate is defined—that is to say, substantially in the direction of the optical axis) becomes superfluous in the marginal region. As a result, the duration of the operation can also be distinctly shortened.

### LIST OF REFERENCE SYMBOLS

10 cornea
10a flap
12 limbus
14 edge (of the flap)
16 articulated part (‘hinge’)
18 focal plane of the laser
18a incision region
20 lateral incision
22 anterior surface (of 10)
24 posterior surface (of 10)
26 sclera
28 suction ring
30 applanation plate
32 openings
34 connection piece
36 duct
38 cavity (vacuum)
40 radiation-opaque body
40a inner edge
42 sealing surface
44 suction-ring contact plate
46 vacuum pump
D1 diameter
D2 thickness
α angle
β angle
K circle
X region
S stroma

1. Apparatus for cutting a tissue part (10a) out of a tissue (10) by means of focused laser radiation, exhibiting the following:
a suction ring (28) which has a sealing surface (42) that is capable of being applied onto a surface (22) of the tissue (10),
devices (34, 36, 46) for generating an underpressure in a cavity (38) that is delimited by the surface (22) of the tissue (10) and by the suction ring, and
an applanation plate (30) that is capable of being pressed against the surface of the tissue (20) for the purpose of shaping,
characterised by
a body (40) that is opaque to the laser radiation, that interacts with the suction ring (28) and with its inner edge (40a) defines an edge (14) of the tissue part (10a).

2. Apparatus according to claim 1, characterised in that the body (40) that is opaque to laser radiation is annular.

3. Apparatus according to one of claims 1 or 2, characterised in that the body (40) that is opaque to laser radiation at least partly absorbs the laser radiation.

4. Apparatus according to one of the preceding claims, characterised in that the body (40) that is opaque to laser radiation rests on a contact plate (44) that with a sealing surface (42) is capable of being brought into contact with the tissue (10).

5. Apparatus according to one of the preceding claims, characterised in that the sealing surface (42) is capable of being brought into contact with the cornea (10) of an eye and in that the inner edge (40a) of the body (40) that is opaque to the laser radiation defines the edge of a flap (10a) that is capable of bring folded upwards in the course of an ophthalmological operation.
